(19) United States
(12) Patent Application Publication
(10) Pub. No.: US 2011/0036787 A1
(43) Pub. Date: Feb. 17, 2011

(54) PARTICLE SEPARATION

(75) Inventors: Mordechai DEUTSCH, Doar-Na Lev HaSharon (IL); Assaf DEUTSCH, Tzfaria (IL)

Correspondence Address: MARTIN D. MOYNIHAN d/b/a PRTSI, INC., P.O. BOX 16446, ARLINGTON, VA, 22215, US

(73) Assignee: Seng Enterprises Ltd., Larnaca (CY)

(21) Appl. No.: 12/854,943

(22) Filed: Aug. 12, 2010

(30) Foreign Application Priority Data
Aug. 12, 2009 (IL) 200359

(51) Int. Cl. B01D 29/66

(52) U.S. Cl. 210/797; 210/409; 210/410; 210/196

(57) ABSTRACT

Apparatus for increasing a concentration of large particles in a mixture which includes the large particles and smaller particles, including a flow chamber, a filtrate accepting chamber, a filtering component separating the flow chamber from the filtrate accepting chamber, the filtering component including a plurality of pores passing therethrough, characterized by further including means for generating a flow of the fluid in the flow chamber, with a flow component in a direction parallel to a surface of the filtering component sufficient to substantially prevent particles from adhering to the filtering component and clogging the pores. A method of increasing a concentration of large particles in a mixture which includes the large particles and smaller particles, including providing a flow chamber and a filtrate accepting chamber separated by a filtering component, the filtering component including a plurality of pores passing therethrough, and placing a fluid with the mixture in the flow chamber, characterized by causing the fluid with the mixture to flow in the flow chamber, with a flow component parallel to a surface of the filtering component sufficient to substantially prevent particles from adhering to the filtering component and clogging the pores, thereby increasing the concentration of the larger particles relative to the concentration of the smaller particles in the mixture. Related apparatus and methods are also described.

### RELATED APPLICATION/S

The present application claims priority from Israel Patent Application No. 200359 filed on Aug. 12, 2009, the contents of which are hereby incorporated by reference.

### FIELD AND BACKGROUND OF THE INVENTION

The present invention, in some embodiments thereof, relates to devices and methods useful in sorting particles, such as living cells, by size. The present invention, in some embodiments thereof, also relates to devices and methods useful in separating particles, such as living cells, of a first size from particles of a second size.

It is often necessary to separate particles of one size from a population of particles of a different size when the particles are found in a fluid. An exceptional challenge of separating particles occurs when the desired particles are present in a very low concentration relative to undesired particles.

Background art includes:

U.S. Pat. No. 4,921,603 to Yen;

U.S. Pat. No. 5,855,782 to Falkenhagen et al;

U.S. Pat. No. 6,177,019 to Castino et al;

U.S. Pat. No. 7,182,858 to Brown et al;

US published patent application number 2008/0156709 of Johnson; and

US published patent application number 2010/0140191 of Antunes Franco et al.

### SUMMARY OF THE INVENTION

Embodiments of a device of the present invention allow, amongst other possibilities, the isolation of larger particles (such as living cells such as circulating tumor cells) from smaller particles (such as living cells such as peripheral blood leucocytes), especially when the relative concentration of the larger particles is significantly lower than that of the smaller particles.

In some embodiments of the present invention there is provided a method of increasing a concentration of large particles in a mixture which includes the large particles and smaller particles, including providing a flow chamber and a filtrate accepting chamber separated by a filtering component, the filtering component comprising a plurality of pores passing therethrough; and placing a fluid with the mixture in the flow chamber, characterized by: causing the fluid with the mixture to flow in the flow chamber, with a flow component parallel to a surface of the filtering component sufficient to substantially prevent particles from adhering to the filtering component and clogging the pores, thereby increasing the concentration of the larger particles relative to the concentration of the smaller particles in the mixture.

According to an aspect of some embodiments of the present invention there is provided apparatus for increasing a concentration of large particles in a mixture which includes the large particles and smaller particles, including a flow chamber, a filtrate accepting chamber, a filtering component separating the flow chamber from the filtrate accepting chamber, the filtering component including a plurality of pores passing therethrough, characterized by further including means for generating a flow of the fluid in the flow chamber, with a flow component in a direction parallel to a surface of the filtering component sufficient to substantially prevent particles from adhering to the filtering component and clogging the pores.

According to some embodiments of the invention, the large particles are cells and the smaller particles are cells.

According to some embodiments of the invention, the means for generating a flow includes a peristaltic pump.

According to some embodiments of the invention, the flow chamber is adapted to form a closed circuit, at least for some of its operational cycle.

According to some embodiments of the invention, the filtrate accepting chamber is adapted to form a closed circuit, at least for some of its operational cycle.

According to some embodiments of the invention, further including a mechanical support for preventing the filtering component from substantially deforming.

According to some embodiments of the invention, the support is a helical support substantially in contact with the filtering component.

According to some embodiments of the invention, the support is a woven tube substantially in contact with the filtering component.

According to some embodiments of the invention, further including a means for generating a pressure differential R=Pflow/Pfiltrate between a pressure of fluid in the flow chamber Pflow and a pressure of fluid in the filtrate accepting chamber Pfiltrate.

According to some embodiments of the invention, further including a pump for adding fluid to the flow chamber, generating a pressure differential R=Pflow/Pfiltrate between a pressure of fluid in the flow chamber Pflow and a pressure of fluid in the filtrate accepting chamber Pfiltrate.

According to some embodiments of the invention, further including a mechanism for adding gas to a pressurizing reservoir connected to the flow chamber, generating a pressure differential R=Pflow/Pfiltrate between a pressure in the flow chamber Pflow and a pressure in the filtrate accepting chamber Pfiltrate.

According to some embodiments of the invention, further including a pump for removing fluid from the filtrate accepting chamber, generating a pressure differential R=Pflow/Pfiltrate between a pressure of fluid in the flow chamber Pflow and a pressure of fluid in the filtrate accepting chamber Pfiltrate.

According to some embodiments of the invention, the pressure differential R does not exceed pressure differentials which are applied to cells in a living body.

According to some embodiments of the invention, the pressure of fluid in the flow chamber Pflow is substantially 1 atmosphere +/−10%.

According to some embodiments of the invention, further including an outer wall which defines a volume of the filtrate accepting chamber.

According to some embodiments of the invention, the means for generating a flow includes a peristaltic pump, and at least a portion of the outer wall which is in contact with the peristaltic pump is substantially flexible.

According to some embodiments of the invention, further including an outer wall which limits a volume of the flow chamber.

According to some embodiments of the invention, the means for generating a flow includes a peristaltic pump, and at least a portion of the outer wall which is in contact with the peristaltic pump is substantially flexible.

According to an aspect of some embodiments of the present invention there is provided a method of increasing a concentration of large particles in a mixture which includes the large particles and smaller particles, including providing a flow chamber and a filtrate accepting chamber separated by a filtering component, the filtering component including a plurality of pores passing therethrough, and placing a fluid with the mixture in the flow chamber, characterized by causing the fluid with the mixture to flow in the flow chamber, with a flow component parallel to a surface of the filtering component sufficient to substantially prevent particles from adhering to the filtering component and clogging the pores, thereby increasing the concentration of the larger particles relative to the concentration of the smaller particles in the mixture.

According to some embodiments of the invention, the large particles are cells and the smaller particles are cells.

According to some embodiments of the invention, the flow of the fluid in the flow chamber includes a substantial flow in a direction parallel to a surface of the filtering component.

According to some embodiments of the invention, the fluid with the mixture flows in a closed circuit, during at least a portion of an operational cycle, thereby continuously increasing the concentration of the larger particles relative to the concentration of the smaller particles in the mixture.

According to some embodiments of the invention, the flows in a closed circuit applies only to the fluid in the flow chamber.

According to some embodiments of the invention, a ratio R of pressure of fluid on the flow chamber side of the filtering component (Pflow) to a pressure of fluid on the filtrate accepting chamber side of the filtering component (Pfiltrate), is caused to be greater than 1.

According to some embodiments of the invention, the ratio R is caused to be greater than one by adding fluid to the flow chamber.

According to some embodiments of the invention, the ratio R is caused to be greater than one by removing fluid from the filtrate accepting chamber.

According to some embodiments of the invention, the ratio R is caused to be greater than one by maintaining such a flow of fluid in the filtrate accepting chamber that the Venturi effect causes a lower pressure in the filtrate accepting chamber than in the flow chamber.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A and 1B are simplified block diagram illustrations of cross sections of a filtering device constructed according to a first example embodiment of the invention;

FIGS. 1C and 1D are simplified block diagram illustrations of cross sections of a filtering device constructed according to a second example embodiment of the invention;

FIG. 2A is a simplified block diagram illustration of a filter constructed according to a third example embodiment of the invention;

FIG. 2B is a simplified illustration of a filtering component, shaped to produce a low pressure section of a filtrate accepting chamber;

FIG. 3 is a simplified block diagram illustration of optional sensors in a filter constructed according to a fourth example embodiment of the invention; and

FIG. 4 is a simplified flow chart illustration of a method of increasing a concentration of large particles in a mixture which includes the large particles and smaller particles according to an embodiment of the present invention.

### DESCRIPTION OF SPECIFIC EMBODIMENTS OF THE INVENTION

The present invention, in some embodiments thereof, relates to devices and methods useful in sorting particles, such as living cells, by size. The present invention, in some embodiments thereof, also relates to devices and methods useful in separating particles, such as living cells, of a first size from particles of a second size.

In one example of filtration, in the blood of living organisms Circulating Tumor Cells (CTCs) may be found in a relatively low concentration, typically 1 such cell in 1 ml of blood. One implementation of isolating CTCs, is an isolation from Peripheral Blood Leucocytes (PBLs), which are much more common, typically 10⁷ PBLs for every CTC. As circulating tumor cells typically have a diameter of about 30 micrometers and peripheral blood leucocytes are smaller, typically having a diameter of up to about 15 micrometers, one might consider concentrating tumor cells by filtration.

In an example of prior art cell filtration, a sample of blood fraction is filtered through a membrane having holes small enough to allow passage of smaller cells such as peripheral blood leukocytes but to prevent the passage of larger cells such as Circulating Tumor Cells (CTCs). A volume of fluid containing a mixture of the cells is placed on one side of a membrane and forced to pass through the membrane.

An example membrane which is used in prior art for attempting such filtration is a Millipore™ membrane filter with appropriate sized pores. Such a membrane typically provides good control over pore sizes, and may be smooth, yet nevertheless suffers from clogging and from cells sticking to the filter material when used for filtration according to prior art techniques.

A first problem occasionally encountered with such a method is to recover the large cells from the membrane, and/or keep the larger cells from clogging the membrane pores. Useful membranes filters have pores of a size which allow entry of the larger cells to such an extent that at least some are stuck in the membrane pores. The problem is exacerbated by the fact that some cell types are flexible and/or deformable. Their flexibility is useful in a living body so they can squeeze between other cells by deforming, but this same feature may cause the larger cells to deform into, and clog up, a filter pore. This problem is avoided in embodiments of the present invention by maintaining a flow of fluid at least partly tangential to the filter membrane, dislodging the larger cells from the membrane pores.

A second problem occasionally encountered with such a method is that many small cells do not pass through the membrane but get stuck to the membrane, so that one rarely gets complete isolation of large cells from small cells. It is generally accepted that filtration by known techniques increases the relative concentration of the large cells relative to the small cells by three orders of magnitude, e.g. from 1 in 10⁷ to 1 in 10⁴, which is often insufficient.

A third problem occasionally encountered with such a method is how to avoid drying of the large cells if too much fluid passes through the filter. This problem is avoided in embodiments of the present invention by adding appropriate fluid, such as, by way of some non-limiting examples: water; saline solution; more cell and fluid mixture for filtration; or even taking post-filtration fluid from a post-filtration side, additionally filtering out the smaller cells, and re-introducing the additionally filtered post-filtration fluid to the as-yet unfiltered side of the filter.

The present invention, in some embodiments thereof, teaches a way to better separate particles by size, for example to isolate large circulating tumor cells from smaller cells such as peripheral blood leucocytes.

The present invention, in some embodiments thereof, includes a method of concentrating large particles in a mix of small particles and large particles, by causing a fluid carrying the mix to flow along a porous membrane, the membrane having holes substantially sized between the small size and the large size. The method includes maintaining a flow of the fluid along the membrane surface.

Maintaining the flow acts to clear both small and large cells which typically stick to the membrane, also keeping the membrane clear from clogging.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

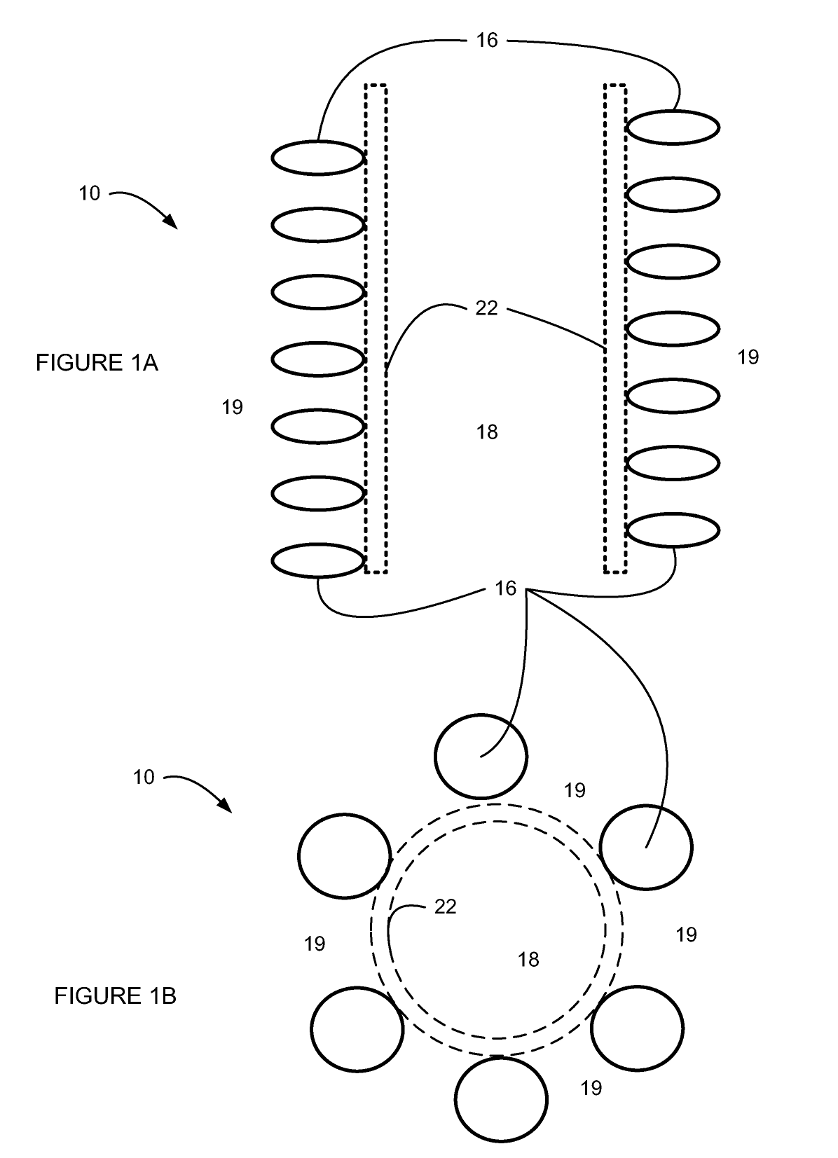

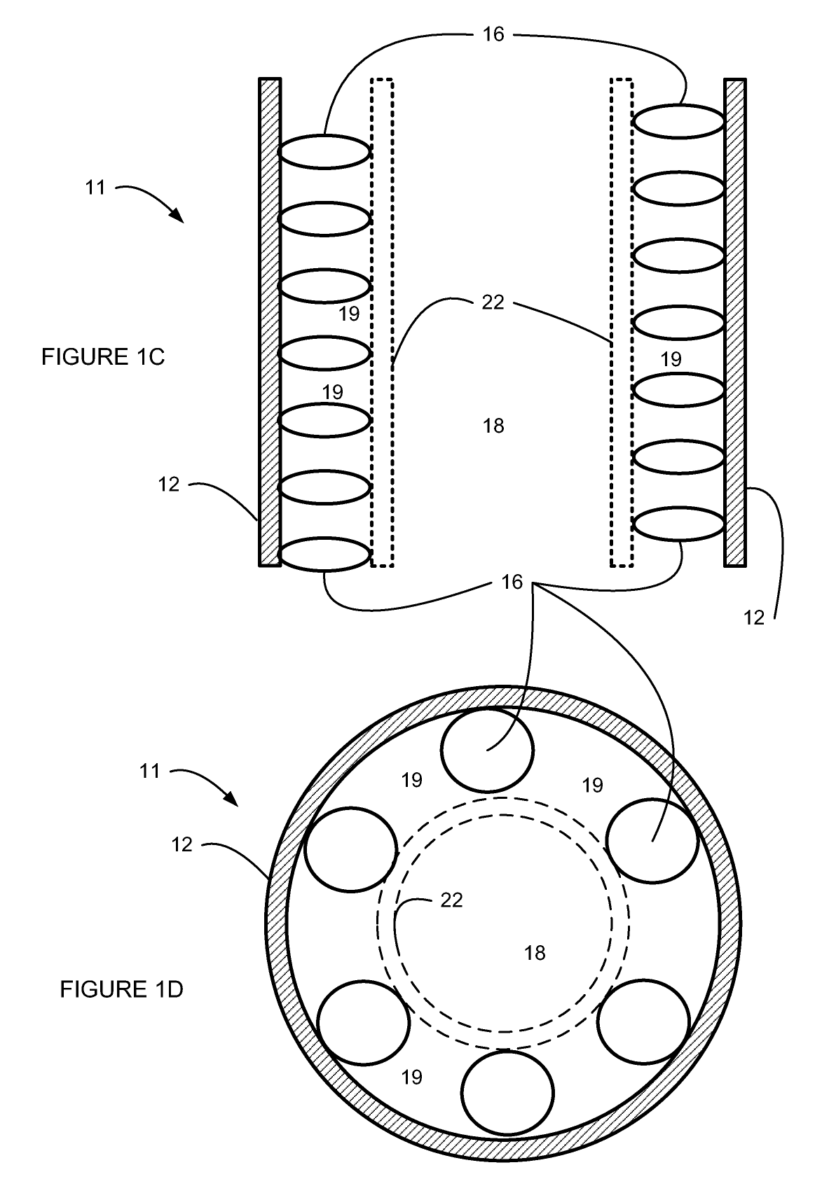

Reference is now made to FIGS. 1A and 1B, which are simplified block diagram illustrations of cross sections of a filtering device 10 constructed according to a first example embodiment of the invention.

The filtering device 10 includes a porous filtering component 22, which includes pores sized so as to substantially let through a first size of particles, such as cells, and substantially block a second, larger, size of particles.

In some embodiments the filtering component 22 is a membrane, optionally a membrane with substantially known filtering pore size. Optionally the pores are substantially uniform in size. Optionally the surface of the filtering component 22 is smooth, to lower a likelihood of particles/cells sticking to the surface, and/or to lower a chance of damage to cells.

The filtering component 22 is supported by a support 16. In the non-limiting example case of the filtering device 10 of FIGS. 1A and 1B, the support 16 is a helical support. FIG. 1A depicts the support 16 as surrounding the porous filtering component 22, thereby providing the support. FIG. 1B depicts the support 16 as being made of six helical strands. The example of FIGS. 1A and 1B is not to be understood as limiting, since a helical support 16 may be made of even a single strand, and any number of strands for the support 16 is contemplated.

The support 16, in other embodiments, does not necessarily have to be helical. Parallel wires may be used for support, or a tube woven of strands, or a perforated tube.

In various embodiments of the invention, the support may be made of polymer materials, such as Polystyrene, Polypropylene, Tygon, Teflon, Silicone, Polyethylene, and so on, and/or woven glass fiber, and/or a stainless steel mesh.

In some embodiments, and especially when the filtering component 22 is a deformable and/or flexible membrane, the support 16 optionally prevents the filtering component 22 from substantially deforming.

The structure of the filtering device 10 produces a flow chamber 18 on one side of the filtering component 22 and a filtrate accepting chamber 19 on another side of the filtering component 22.

Some technical observations on the filtering device 10 of FIGS. 1A and 1B are now made, which also apply to other embodiments of the present invention.

In order to promote transfer of the first, smaller, sized particles through the membrane, a pressure differential may be produced between a flow chamber 18 on one side of the filtering component 22 and a filtrate accepting chamber 19. The pressure differential is not necessary—even without a pressure differential, the smaller particles will eventually migrate from the flow chamber 18 to the filtrate accepting chamber 19. If the smaller particles, which might migrate back to the flow chamber 18, are evacuated from the filtrate accepting chamber 19, the concentration of the larger particles left in the flow chamber 18 will rise.

Nevertheless, in some embodiments of the invention, a pressure differential is optionally produced between the flow chamber 18 and the filtrate accepting chamber 19.

In some embodiments of the invention, a ratio R of pressure of fluid on the flow chamber 18 side of the filtering component 22 (Pflow) to a pressure of fluid on the filtrate accepting chamber 19 side of the filtering component 22 (Pfiltrate), is caused to be greater than 1.

In some embodiments of the invention, especially when intended for use to separate cells without harming the cells, the pressure differential R does not exceed pressure differentials which are applied to cells in a living body. It is noted that cells do sometimes undergo pressure differentials, and some cells, such as, by way of a non-limiting example, red blood cells, are capable of flexing in order to pass through narrow passages.

In some embodiments of the invention, the ratio R is caused to be greater than one by measuring a pressure in the flow chamber, and maintaining the pressure in the flow chamber higher than a set level.

In some embodiments of the invention, the ratio R is caused to be greater than one by measuring a pressure in the flow chamber and in the filtrate accepting chamber, and maintaining the pressure in the flow chamber higher than the pressure in the filtrate accepting chamber.

In some embodiments of the invention, the ratio R is caused to be greater than one by measuring a pressure in the filtrate accepting chamber, and maintaining the pressure in the filtrate accepting chamber lower than a set level.

In some embodiments of the invention, the ratio R is caused to be greater than one by measuring a pressure in the flow chamber and in the filtrate accepting chamber, and maintaining the pressure in the filtrate accepting chamber lower than the pressure in the flow chamber.

In some embodiments of the invention, the ratio R is caused to be greater than one by adding fluid to the flow chamber.

In some embodiments of the invention, the ratio R is caused to be greater than one by adding gas to a pressurizing reservoir (not shown) connected to the flow chamber.

In some embodiments of the invention, the ratio R is caused to be greater than one by removing fluid from the filtrate accepting chamber.

In some embodiments of the invention, an appropriate fluid, such as, by way of some non-limiting examples, water, or saline solution, or more cell and fluid mixture for filtration, is added to the flow chamber 18, to maintain the ratio R and/or prevent drying of cells in the flow chamber 18.

In some embodiments of the invention, the filtering component 22 is a permeable membrane. In embodiments the membrane is a filtering membrane known in the art of filtering. In embodiments the membrane is coated with a layer preventing adhesion of cells thereto.

In embodiments, the filtering device 10 comprises one or more pipes, e.g., of glass or the like, such as a glass outer envelope to the filtrate accepting chamber 19, and/or a glass support 16.

In some implementations of the filtering device 10, the filtering component is adapted, for example by selection of pore size and pressure differential as further detailed below, and by selection of a smooth filter material, to work best with large particles which are substantially smooth, like cells.

In some implementations of the filtering device 10, the filtering component is adapted, for example by selection of pore size and pressure differential as further detailed below, to work best with large particles which are relatively uniform in shape, like cells.

In some implementations of the filtering device 10, the filtering component is adapted to work best with large particles which are all of substantially similar size, like a specific type of cell.

In some implementations of the filtering device 10, the filtering component is adapted to work best with large particles which have a diameter of at least about 10%, of at least about 20%, of at least about 30%, of at least about 40%, of at least about 50%, of at least about 60%, of at least about 70%, of at least about 80%, at least about 90% and even of at least about 100% (twice) that of the smaller particles.

In some implementations of the filtering device 10, the filtering component is adapted to work best with large particles which have a volume of at least about 1.2 times, of at least about 1.4 times, of at least about 1.7 times, of at least about 2 times, of at least about 2.2 times, of at least about 2.5 times, of at least about 2.9 times, of at least about 3.2 times, at least about 3.6 times and even of at least about 4 times that of the smaller particles.

In some implementations of the filtering device 10, the filtering component is adapted to work best with large particles which are circulating tumor cells (CTCs).

In some implementations of the filtering device 10, the filtering component is adapted to work best with large particles which have a diameter of at least about 10, at least about 15, at least about 20 and even at least about 25 micrometers. In embodiments, the large particles have a diameter of no more than about 100, no more than about 90, no more than about 80 and even no more than about 70 micrometers.

In some implementations of the filtering device 10, the filtering component is adapted, by selecting pore size and/or pressure differential, as further explained below, to work best with large particles which have a volume of at least about 4200, at least about 14100, at least about 33500 and even at least about 65400 micrometer³. In embodiments, the large particles have a volume of no more than about 4,189,000, no more than about 3,054,000, no more than about 2,145,000 and even no more than about 1,437,000 micrometer³.

Embodiments of the present invention enable a separation of larger particles from smaller particles. Discussed herein are some embodiments of devices suitable in implementing the teachings of the present invention for isolating larger cells from smaller cells in a fluid (liquid, e.g. whole blood). In some cases the larger cells are present in a significantly lower concentration, and specifically separating circulating tumor cells (CTCs) from whole blood.

It is noted that some embodiments of the present invention do not isolate only one type of the larger particles, but rather that the isolated particles are a subpopulation of particles from an original population of particles, where the subpopulation includes particles that are greater than or equal to a certain size. Further, it is expected since some aspects of the present invention are based on a probability of a smaller particle passing through a channel of a separation wall, that in some embodiments there will be present particles smaller than the certain size, but in a relative concentration which may be inversely proportional to the size of the particle, that is, relatively fewer relatively smaller particles.

It is noted that the longer application of the present invention is applied to a given sample, the lower the relative concentration of the smaller articles will be.

It is noted that some embodiments of the present invention, when compared to prior art filtering techniques for separating CTCs from blood, allow for the use of a relatively small surface area of filtering component for a relatively large sample volume, while providing a relatively high degree of separation of different sized particles.

It is noted that in embodiments of the present invention, a passage of fluid through the filtering component 22 produces currents in proximity to the filtering component 22, which reduce a chance that a particle adheres to the filtering component 22.

Reference is now made to FIGS. 1C and 1D, which are simplified block diagram illustrations of cross sections of a filtering device 11 constructed according to a second example embodiment of the invention.

The filtering device 11 includes a porous filtering component 22, which includes pores sized so as to substantially let through a first size of particles, and substantially block a second, larger, size of particles. The filtering component 22 is supported by a support 16. In the non-limiting example case of the filtering device 11 of FIGS. 1C and 1D, the support 16 is a helical support. A helical support provides support to the filtering component 22 without obstructing flow, and when tightly fitted to the filtering component 22 and the outer wall 12, makes a longer fluid path in the filtrate accepting chamber 19 than in the flow chamber 18. FIG. 1C depicts the support 16 as surrounding the porous filtering component 22, thereby providing the support. FIG. 1D depicts the support 16 as being made of six helical strands. The example of FIGS. 1C and 1D is not to be understood as limiting, since a helical support 16 may be made of even a single strand, and any number of strands for the support 16 is contemplated.

The structure of the filtering device 11 produces a flow chamber 18 on one side of the filtering component 22 and a filtrate accepting chamber 19 on another side of the filtering component 22.

The bore of the filtering component 22 defines the flow chamber 18. The generally tubular volume between an outer wall 12 and the filtering component 22, which is partially taken up by the support 16 defines the filtrate accepting chamber 19.

The outer wall 12 envelops the support 16, limiting an extent of the filtrate accepting chamber 19.

In some embodiments of the invention, the outer wall 12 is rigid or semi-rigid, and optionally provides further mechanical support to the support 16. In some embodiments of the invention the support 16 of FIGS. 1C and 1D is more flexible than the support 16 of FIGS. 1A and 1B, and optionally the outer wall 12 provides mechanical support.

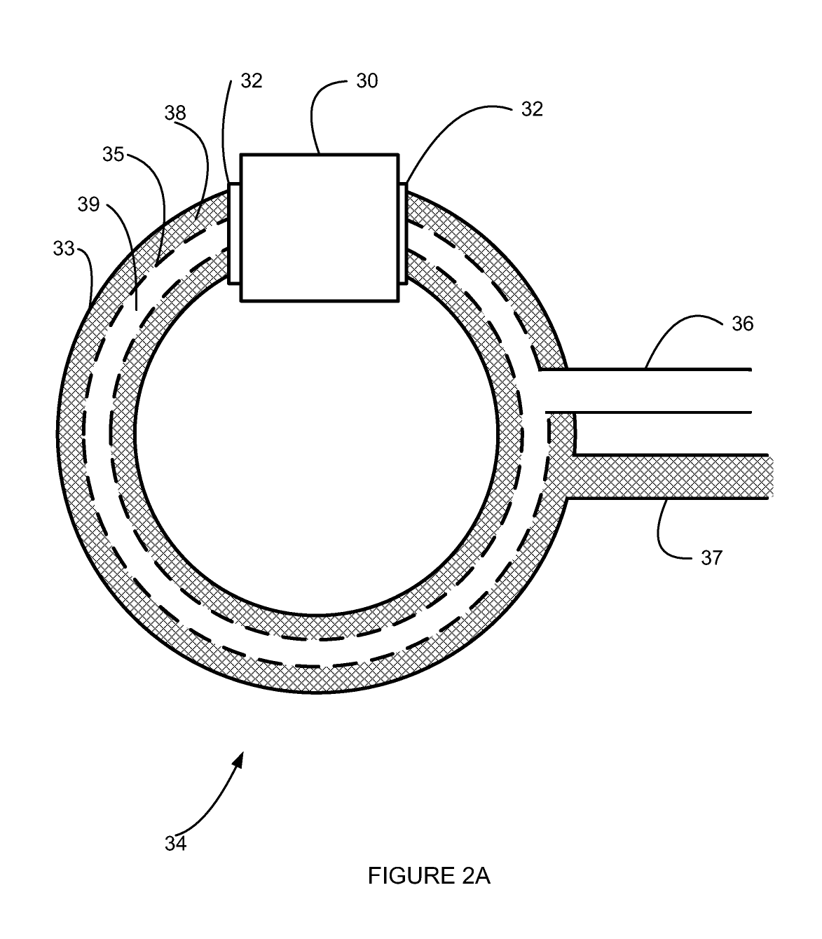

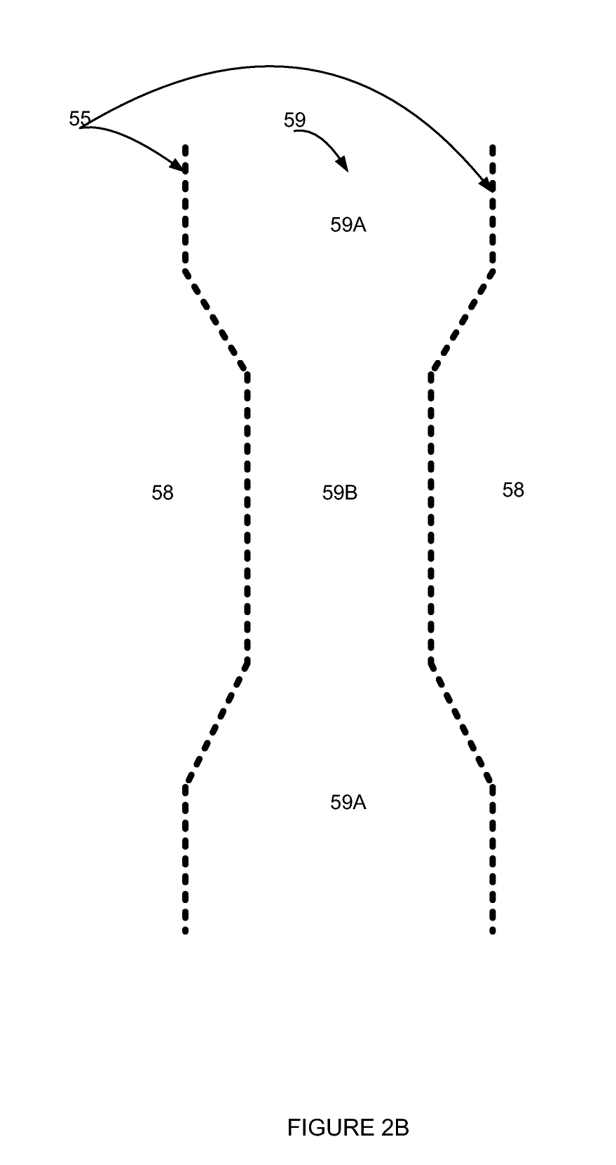

It is noted that in some embodiments of the filtering device of FIGS. 1A and 1B, as well as filtering devices described with references to later FIGS. 1C, 1D, and 2, the flow chamber and the filtrate accepting chamber may be exchanged relative to what is depicted in the drawings. That is, for example, with reference to FIGS. 1A and 1B, the flow chamber 18 may be external to the filtering component 22, and the filtrate accepting chamber 19 may be internal to the filtering component 22. In such a case the support 16 is optionally placed on the filtrate accepting chamber 19 side of the filtering component 22, so as not to obstruct fluid flow, and/or so as to provide support against an optional pressure differential.

Some non-limiting technical details which are contemplated for the example embodiment of FIGS. 1C and 1D are include below.

The outer wall 12 is optionally made of stainless steel, having an inner diameter of 5 mm, and 2 mm thick walls. Stainless steel is a material which is not allergenic, easy to keep clean, relatively inexpensive, provides stiff support, and withstands pressure which a separation of cells may exert.

The filtering component 22 optionally made of a 10 micrometer thick polycarbonate membrane, such as produced by MilliPore, having substantially uniformly sized and shaped 20 micrometer diameter perforations. The perforations are optionally produced by ion blasting.

The support 16 optionally made of a braided tube of 0.02 mm stainless steel strands, the braided tube having an inner diameter substantially the same as that of the filtering component 22. Optionally, the braided tube may also have an outer diameter substantially the same as that of the outer wall 12.

An example use of the example embodiment of FIGS. 1C and 1D is now described. The description may be relevant to other example embodiments of the invention.

In the example, a volume of whole blood or blood fraction is introduced into and allowed to flow through the flow chamber 18 at a pressure (Pflow) higher than the pressure (Pfiltrate) in the filtrate accepting chamber 19. Since the pressure in the flow chamber 18 is higher than the pressure in the filtrate accepting chamber 19, fluid passes through perforations in the filtering component 22. Particles, such as blood cells and/or peripheral blood leucocytes, which are smaller than the perforations, which are for example about 20 micrometers in diameter, pass through the filtering component 22, while larger particles remain in the flow chamber 18.

Larger particles which incidentally rest in imperfections in the filtering component 22 or are otherwise attracted to an inner surface of the filtering component 22 are dislodged by a flow of fluid.

Some of the smaller particles which may not yet have passed through the perforations, but stick to or near the inner surface, are dislodged by the flow of fluid. It is noted that laminar flow of the fluid, which might carry smaller particles past the inner surface of the filtering component 22 without allowing contact therewith, is substantially prevented, or at least substantially lessened, due to a continuous flow of fluid through the perforations.

In some applications, the flow of fluid through the perforations applies a force on the particles towards a surface of the filtering component 22. In some applications, due to the greater momentum of the larger particles relative to the smaller particles, the smaller particles are more quickly and more often brought in contact with the filtering component 22, while larger particles which do encounter the surface of the filtering component 22 do so more forcefully and tend to bounce away.

A given volume of fluid flowing further and further through the flow chamber 18 of the filtering device 10 includes a higher and higher relative concentration of larger particles and a lower and lower relative concentration of smaller particles. Due to the flow, larger particles are substantially prevented from sticking to the surface of the filtering component 22 and are thus substantially all recoverable by recovery of the fluid inside the flow chamber 18. Due to the flow, smaller particles are substantially prevented from coming to rest on the surface of the filtering component 22 and are rather forced, given enough time or enough length of the filtering device 10, to pass into the filtrate accepting chamber 19.

Stringing More Than One Filter in a Row

It is noted that it is possible to string more than one filter, such as the filter 11 depicted in FIG. 1D, in a row. A first filter optionally has a filtering component for filtering out a first size of cells. Output from either the flow chamber or the filtrate chamber of the first filter may enter a flow chamber of a second filter.

In some embodiments, two filters in a row, with equal sized pores, are optionally strung together, with output of a flow chamber of a first filter being provided to a flow chamber of a second filter, achieving a higher separation between different sized cells.

In some embodiments, two filters are strung in a row, with output of a flow chamber of a first filter being provided to a flow chamber of a second filter, and with pores of the first filter being larger than pores of the first filter. The above setup achieves a separation between smallest cells in the filtrate chamber of the first filter, larger sized cells in the filtrate chamber of the second filter, and largest sized cells in a flow chamber of the second of the second filter.

Other combinations of stringing together more than two filters, and connecting outputs of flow chambers to filtrate chambers or outputs of filtrate chambers to flow chambers are also contemplated.

Reference is now made to FIG. 2A, which is a simplified block diagram illustration of a filtering device 34 constructed according to a third example embodiment of the invention.

The filtering device 34 includes a porous filtering component 35 having a substantially toroidal shape, within an outer wall 33, also having substantially toroidal shape. The filtering component 35 may or may not be supported by a support as in FIGS. 1A-1D. FIG. 2A depicts the filtering device 34 without the support, for simplicity of depiction.

The structure of the filtering device 34 produces a flow chamber 38 on one side of the filtering component 35 and a filtrate accepting chamber 39 on another side of the filtering component 35.

The outer wall 33 envelops the filtering component 35 leaving some spacing between them, which defines an extent of the flow chamber 38.

The flow chamber 38 has at least one mixture supply tube 37 connected, allowing fluid to flow into, or out of, the flow chamber 38.

The filtrate accepting chamber 39 has at least one filtrate tube 36 connected, allowing fluid to flow out of, or into, the filtrate accepting chamber 39.

A flow generator 30 is placed somewhere along, and functionally associated with, the toroid of the filtering device 34, enveloping the outer wall 33.

In a non-limiting example implementation of the example embodiment of FIG. 2A the flow generator 30 is a peristaltic pump, and at least a portion 32 of the outer wall 33 is flexible, at least in a section which is within and/or extends slightly beyond the flow generator 30. The outer wall 33 may be made, by way of a non-limiting example, of fiber-reinforced Tygon® tubing.

It is noted that in some embodiments, when the flow generator 30 is a peristaltic pump, the flow chamber 38 is external to and envelopes the filtrate accepting chamber 39. The peristaltic pump produces a main flow generation effect in the external chamber, which is the flow chamber 38. in some embodiments, the peristaltic pump produces flow in both the external chamber and the internal chamber, optionally by virtue of both the outer wall 33 and the filtering component 35 being flexible, at least within the peristaltic pump, and it does not matter whether the flow chamber 38 envelopes the filtrate accepting chamber 39, or the filtrate accepting chamber 39 envelopes the flow chamber 38.

Some example applications of the example embodiment of FIG. 2A are now described. The description may be relevant to other example embodiments of the invention.

A sample of fluid including the particles to be sorted is introduced into the flow chamber 38 through the mixture supply tube 37.

Various flow and pressure regimes may be maintained in the above-mentioned example applications.

Fluid which passes from the flow chamber 38 through the filtering component 35 into the filtrate accepting chamber 39 may optionally be replenished through the mixture supply tube 37.

Filtrate, including smaller particles, may optionally be removed from the filtrate accepting chamber 39 through the filtrate tube 36. A continuous circular flow is optionally generated around the toroidal shaped flow chamber 38 by the flow generator 30.

When fluid is optionally continuously removed from the filtrate accepting chamber 39 through the filtrate tube 36 a desired pressure differential may be generated, limited only by technical features such as the strength of the materials used, and of the membrane making up the filtration component 35.

When fluid is optionally continuously introduced into the flow chamber 38 through the mixture supply tube 37 a desired pressure differential may be generated, limited only by technical features such as the strength of the materials used, and of the membrane making up the filtration component 35.

In some embodiments of the invention, an appropriate fluid, such as, by way of some non-limiting examples, water, or saline solution, or more cell and fluid mixture for filtration, is added to the flow chamber 38, to maintain the ratio R and/or prevent drying of cells in the flow chamber 38.

Fluid may also be optionally both continuously removed from the filtrate accepting chamber 39 through the filtrate tube 36 and continuously introduced into the flow chamber 38 through the mixture supply tube 37, achieving the desired pressure differential.

In some applications, a direct control of the pressure Pfiltrate in the filtrate accepting chamber 39 is optionally effected, by measuring and maintaining a desired pressure, rather than by indirectly controlling the pressure by removing or adding fluid.

In some applications, a direct control of the pressure Pflow in the flow chamber 38 is optionally effected, by measuring and maintaining a desired pressure, rather than by indirectly controlling the pressure by adding or removing fluid.

In some applications, a direct control of both the pressure in the flow chamber 38 and in the filtrate accepting chamber 39 are optionally effected.

In some applications, a rate of separation is expected to increase with a greater pressure differential R (=Pflow/Pfiltrate). In some applications it is preferred that R be as large as possible.

It is noted that since the flow through the filtering device 34 is in a closed circuit, the filtering device may be operated for as long as desired, to substantially achieve as great a degree of separation as desired. R may therefore have different values, and a length of time taken for the separation may affect a final degree of separation. In some applications, typical values for R are from about 1 atmosphere, to no more than about 3 atmospheres, no more than about 2 atmospheres, and even no more than about 1.5 atmospheres.

The pressure differential may optionally be induced by either application of pressures or differences in flow velocities, or a combination of both.

In some embodiments, a desired R is achieved by ensuring a faster flow past the filtrate accepting chamber surface of a filtering component than past the flow chamber surface. In accordance with the Bernoulli principle, a pressure differential is achieved.

In some embodiments the flow speed differential may be maintained, for example, by providing constrictions in the filtrate accepting chamber which produce a Venturi effect.

The desired ratio R, such as, for example, an R>1, may be caused by maintaining such a flow of fluid in the filtrate accepting chamber that the Venturi effect causes a lower pressure in the filtrate accepting chamber than in the flow chamber.

For separation from cell-containing fluids, it is generally preferred that the pressure of fluid introduced through the mixture supply tube 37 be about 1 atmosphere, so as not to adversely affect cells in the fluid.

It is noted that while the flow chamber 38 has been described as a closed circuit, for some embodiments, it optionally forms the closed circuit, for one portion of its operational cycle, and opens for accepting a fluid containing a mixture to be separated during another portion of its operational cycle.

Similarly, in some embodiments, the filtrate accepting chamber 39 may optionally be operated as a closed circuit for one portion of its operational cycle, and opens for withdrawing the filtrate during another portion of its operational cycle.

In the embodiment discussed above, the flow chamber 38 is substantially toroidal and defines a closed circuit. In some embodiments of the present invention, the flow chamber is not a closed circuit, and may be, by way of a non-limiting example, linear, curved, and spiral. The flow chamber 38 may optionally have a beginning, substantially in proximity of a fluid inlet and optionally have an end, substantially in proximity of a fluid outlet. The filtrate accepting chamber 39 may optionally have a beginning, substantially in proximity of a fluid inlet and optionally have an end, substantially in proximity of a fluid outlet.

In some of the applications described above, a desired pressure ratio R greater than 1 is achieved by forcing fluid into the flow chamber while removing the fluid from the filtrate accepting chamber. In some embodiments, for example embodiments where the flow chamber is not a closed circuit, a desired R is achieved by forcing fluid into both the flow chamber and the filtrate accepting chamber but at a higher pressure into the flow chamber than into the filtrate accepting chamber. In some embodiments, for example as described above for separating different sized-cells, the types of fluid include isotonic liquids, saline, and PBS.

In embodiments, for example embodiments where the flow chamber is not a closed circuit, a desired R is achieved by ensuring a faster flow past the filtrate chamber surface of a filtering component than past the flow chamber surface, in accordance with the Bernoulli principle. The flow speed differential may be maintained, for example, by providing constrictions in the filtrate accepting chamber which produce a Venturi effect, as is described in more detail below, with reference to FIG. 2B.

It is generally preferred that the perforations through the filtering component be of a relatively uniform size. That said, in some embodiments, the perforations are non-uniformly sized.

Reference is now made to FIG. 2B, which is a simplified illustration of a filtering component 55, shaped to produce a low pressure section 59B of a filtrate accepting chamber 59.

FIG. 2B depicts on constriction in the filtering component 55, which shapes the filtrate accepting chamber 59, as well as optionally affecting a shape of a flow chamber 58. A flow is maintained in the filtrate accepting chamber 59, and the Venturi affect in presence of flow determines that a volume 59B in the filtrate accepting chamber 59, where the filtrate accepting chamber 59 is narrower and the flow is faster, will have lower pressure than another volume 59A, where the filtrate accepting chamber 59 is wider and the flow is slower. The pressure in the flow chamber 58 may be substantially equal to, or higher than, the pressure in the filtrate accepting chamber 59, yet the pressure in the volume 59B of the filtrate accepting chamber 59 will be lower than the pressure in the flow chamber 58.

It is noted that several constrictions to the filtrate accepting chamber 59 may be strung in series, producing several section with lower pressure.

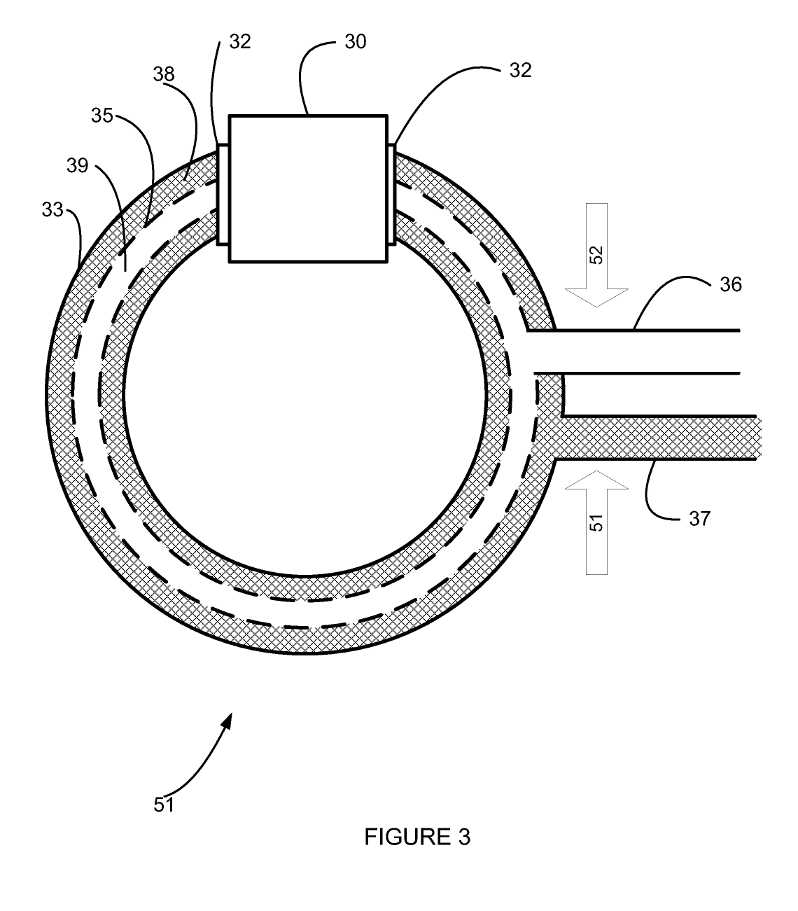

It is noted that the constrictions may optionally be included in straight-line filters, such as depicted in FIGS. 1A, 1B, 1C, and 1D, and may optionally be included in circular filters, such as depicted in FIGS. 2A and 3.

Sensors and Controllers

Reference is now made to FIG. 3, which is a simplified block diagram illustration of optional sensors in a filter 51 constructed according to a fourth example embodiment of the invention.

FIG. 3 is depicted in order to depict an example placement of sensors in an filter 51 including parts similar to the example embodiment depicted in FIG. 2A. The filter 51 includes a porous filtering component 35, a flow chamber 38, a filtrate accepting chamber 39, an outer wall 33 a mixture supply tube 37 a filtrate tube 36 a flow generator 30 and a flexible portion 32 of the outer wall 33, all, by way of a non-limiting example, similar to the parts described with reference to FIG. 2A.

A sensor (not shown) for measuring pressure at a flow chamber 38 may optionally be placed at a location 51 on the mixture supply tube 37. The pressure sensor at the mixture supply tube 37 may optionally be connected to a first controller (not shown) for adding or removing fluid, to control the pressure in the flow chamber 38.

A sensor (not shown) for measuring pressure at a filtrate accepting chamber 39 may optionally be placed at a location 52 on the filtrate tube 36. The pressure sensor at the filtrate tube 36 may optionally be connected to a second controller (not shown) for adding or removing fluid, to control the pressure in the filtrate accepting chamber 39.

The sensors measuring the pressure at the flow chamber 38 and the filtrate accepting chamber 39 may both be connected to a third controller (not shown), which may optionally compare the pressures and control the above-mentioned pressure ratio R.

Selection of Filter Pore Size and of Pressure

Since, as mentioned above, some cell types are flexible and/or deformable, pressure in the flow chamber 38 may push large cells through the filter pores. A judicious selection of pore size and pressure can eliminate such a deficiency in blocking the large cells.

In some embodiments of the invention, a filter pore size is selected to be bigger than the smaller cells, and smaller than the larger cells, so that the smaller cells pass through without substantial hindrance, and the larger cells are blocked.

In some embodiments of the invention, the filter pore size may be selected to be equal to or smaller than the smaller cells, yet at the pressure applied, the smaller cells still pass through the pores without substantial hindrance, while the larger cells are blocked.

When a cell filtration application is defined, that is, when a larger cell type is defined, and its size is known and/or measured, and a smaller type of cell or cells are defined and their sizes are known and/or measured, a pore size is optionally selected, as described above.

Experimenting with filtration at several pressure differentials provides information on what pore size/pressure differential combination produces separation, and optionally provides information on how long the separation process lasts. Once the experiments are done, the pore size/pressure differential combination suitable for a larger cell type/smaller cell type(s) is known, and may optionally be used over and over again.

Detection of Sufficient Separation

In some embodiments of the invention, a sensor (not shown) may optionally be placed to measure cell density in the flow chamber 38. In some embodiments of the invention, the cell density sensor may be an optical density sensor, measuring optical density which correlates to cell density. For cell filtration scenarios as described above, where a ratio of larger cells to smaller cells may be 1:10⁷, it is the smaller cells which produce the optical density, and a reduction in density of the smaller cells is an optional indicator of a changing ratio. When the cell density is measured at a desired level lower than the initial cell density, the ratio may have changed sufficiently, and the cell filtration process may be stopped.

In some embodiments of the invention, a sensor (not shown) may optionally be placed to measure cell density in the filtrate chamber 39. In some embodiments of the invention, the cell density sensor may be an optical density sensor, measuring optical density which correlates to cell density. When the cell filtration starts, cell density at the filtrate chamber 39 rises to a certain level. Optionally fluid containing an abundance of smaller cells is removed from the filtrate chamber 39. After some time, a lowering of the cell density in the filtrate chamber 39 is measured. When the cell density is measured at a desired level lower than the initial cell density, the cell filtration process may be stopped.

In some embodiments of the invention, the detection of sufficient separation automatically stops the cell filtration process.

Stringing More Than One Filter in a Row

It is noted that it is possible to string more than one filter, even filtering devices such as such as the filter devices 34 and 51 depicted in FIGS. 2A, and 3, in a row. An output from a first filter may optionally be input into a second filter, even when the first filter initially operates in a closed loop, and later produces a filtered output. Output from either the flow chamber or the filtrate chamber of the first filter may enter a flow chamber of a second filter.

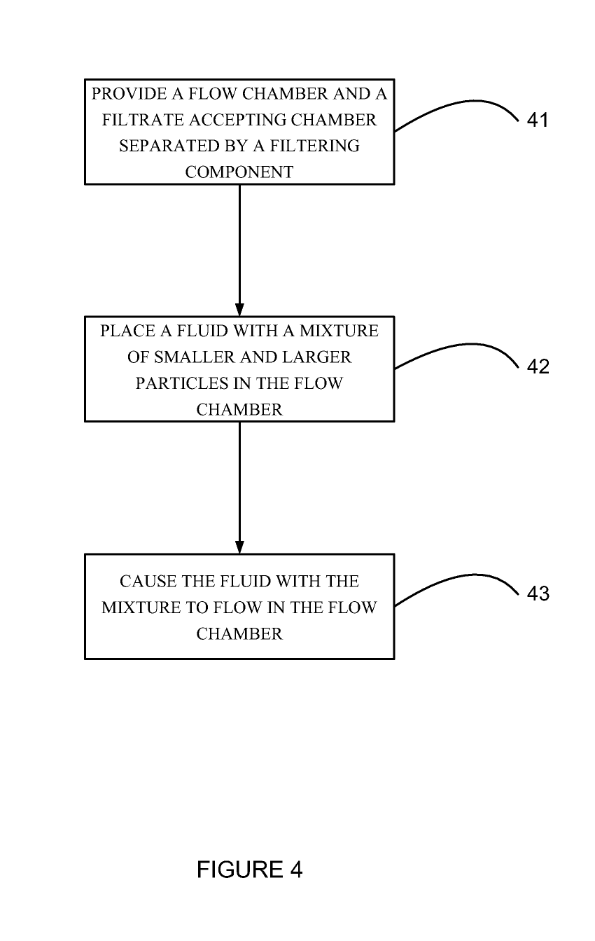

Reference is now made to FIG. 4, which is a simplified flow chart illustration of a method of increasing a concentration of large particles in a mixture which includes the large particles and smaller particles according to an embodiment of the present invention.

First, the conditions under which the increasing happens are provided, that is, a flow chamber and a filtrate accepting chamber, separated by a filtering component, are provided (41). The filtering component optionally has a plurality of pores passing therethrough, for filtering.

A fluid with the mixture of the large particles and the smaller particles is placed in the flow chamber (42).

The method is characterized by causing the fluid with the mixture to flow in the flow chamber (43).

The flow of the fluid in the flow chamber is characterized by having a flow component parallel to a surface of the filtering component sufficient to substantially prevent particles from adhering to the filtering component and clogging the pores.

The flow causes at least some of the members of a subpopulation of smaller particles to pass into the filtrate accepting chamber through the filtering component, while the filtering component prevents the passage of members of the subpopulation of larger particles to pass into the filtrate accepting chamber, and while the flow substantially prevents particles from adhering to the filtering component and clogging the pores,

The filtering out of some of the smaller particles from the mixture with the larger particles causes an increase in the concentration of the larger particles relative to the concentration of the smaller particles in the mixture.

It is expected that during the life of a patent maturing from this application many relevant filtering components and membranes will be developed and the scope of the term filtering components is intended to include all such new technologies a priori.

As used herein the term “about” refers to ±10%.

The terms “comprising”, “including”, “having” and their conjugates mean “including but not limited to”.

The term “consisting of” is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as an “example” or as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. Apparatus for increasing a concentration of large particles in a mixture which includes the large particles and smaller particles, comprising:
a flow chamber;
a filtrate accepting chamber;
a filtering component separating the flow chamber from the filtrate accepting chamber, the filtering component comprising a plurality of pores passing therethrough;
characterized by further including means for generating a flow of the fluid in the flow chamber, with a flow component in a direction parallel to a surface of the filtering component sufficient to substantially prevent particles from adhering to the filtering component and clogging the pores.

2. The apparatus of claim 1 in which the large particles are cells and the smaller particles are cells.

3. The apparatus of claim 1 in which the means for generating a flow comprises a peristaltic pump.

4. The apparatus of claim 1 in which the flow chamber is adapted to form a closed circuit, at least for some of its operational cycle.

5. The apparatus of claim 1 in which the filtrate accepting chamber is adapted to form a closed circuit, at least for some of its operational cycle.

6. The apparatus of claim 1, and further comprising a mechanical support for preventing the filtering component from substantially deforming.

7. The apparatus of claim 6 in which the support is a helical support substantially in contact with the filtering component.

8. The apparatus of claim 6 in which the support is a woven tube substantially in contact with the filtering component.

9. The apparatus of claim 6 and further comprising a means for generating a pressure differential R=Pflow/Pfiltrate between a pressure of fluid in the flow chamber Pflow and a pressure of fluid in the filtrate accepting chamber Pfiltrate.

10. The apparatus of claim 6 and further comprising a pump for adding fluid to the flow chamber, generating a pressure differential R=Pflow/Pfiltrate between a pressure of fluid in the flow chamber Pflow and a pressure of fluid in the filtrate accepting chamber Pfiltrate.

11. The apparatus of claim 6 and further comprising a mechanism for adding gas to a pressurizing reservoir connected to the flow chamber, generating a pressure differential R=Pflow/Pfiltrate between a pressure in the flow chamber Pflow and a pressure in the filtrate accepting chamber Pfiltrate.

12. The apparatus of claim 6 and further comprising a pump for removing fluid from the filtrate accepting chamber, generating a pressure differential R=Pflow/Pfiltrate between a pressure of fluid in the flow chamber Pflow and a pressure of fluid in the filtrate accepting chamber Pfiltrate.

13. The apparatus of claim 9 in which the pressure differential R does not exceed pressure differentials which are applied to cells in a living body.

14. The apparatus of claim 9 in which the pressure of fluid in the flow chamber Pflow is substantially 1 atmosphere +/−10%.

15. The apparatus of claim 1, and further comprising an outer wall which defines a volume of the filtrate accepting chamber.

16. The apparatus of claim 15 in which the means for generating a flow comprises a peristaltic pump, and at least a portion of the outer wall which is in contact with the peristaltic pump is substantially flexible.

17. The apparatus of claim 1, and further comprising an outer wall which limits a volume of the flow chamber.

18. The apparatus of claim 17 in which the means for generating a flow comprises a peristaltic pump, and at least a portion of the outer wall which is in contact with the peristaltic pump is substantially flexible.

19. A method of increasing a concentration of large particles in a mixture which includes the large particles and smaller particles, comprising:
providing a flow chamber and a filtrate accepting chamber separated by a filtering component, the filtering component comprising a plurality of pores passing therethrough; and
placing a fluid with the mixture in the flow chamber,
characterized by:
causing the fluid with the mixture to flow in the flow chamber, with a flow component parallel to a surface of the filtering component sufficient to substantially prevent particles from adhering to the filtering component and clogging the pores,
thereby increasing the concentration of the larger particles relative to the concentration of the smaller particles in the mixture.

20. The method of claim 19 in which the large particles are cells and the smaller particles are cells.

21. The method of claim 19 in which the flow of the fluid in the flow chamber comprises a substantial flow in a direction parallel to a surface of the filtering component.

22. The method of claim 19 in which the fluid with the mixture flows in a closed circuit, during at least a portion of an operational cycle, thereby continuously increasing the concentration of the larger particles relative to the concentration of the smaller particles in the mixture.

23. The method of claim 22 in which the flows in a closed circuit applies only to the fluid in the flow chamber.

24. The method of claim 19 in which a ratio R of pressure of fluid on the flow chamber side of the filtering component (Pflow) to a pressure of fluid on the filtrate accepting chamber side of the filtering component (Pfiltrate), is caused to be greater than 1.

25. The method of claim 24 in which the ratio R is caused to be greater than one by adding fluid to the flow chamber.

26. The method of claim 24 in which the ratio R is caused to be greater than one by removing fluid from the filtrate accepting chamber.

27. The method of claim 24 in which the ratio R is caused to be greater than one by maintaining such a flow of fluid in the filtrate accepting chamber that the Venturi effect causes a lower pressure in the filtrate accepting chamber than in the flow chamber.
